(19) United States
(12) Patent Application Publication
(10) Pub. No.: US 2025/0281217 A1
(43) Pub. Date: Sep. 11, 2025

(54) SYSTEMS AND METHODS FOR PULSED FIELD ABLATION USING LOW VOLTAGE, LONG DURATION PULSES

(71) Applicant: St. Jude Medical, Cardiology Division, Inc., St. Paul, MN (US)

(72) Inventors: Lakshya Mittal, Dayton, MN (US); Gregory K. Olson, Elk River, MN (US)

(21) Appl. No.: 19/072,365

(22) Filed: Mar. 6, 2025

Related U.S. Application Data
(60) Provisional application No. 63/562,504, filed on Mar. 7, 2024.

(51) Int. Cl. A61B 18/00

(52) U.S. Cl. CPC A61B 18/00; A61B 2018/00267; A61B 2018/00613; A61B 2018/00761; A61B 2018/00767

(57) ABSTRACT

Systems and methods for electroporation are provided. An electroporation system includes a catheter assembly including a first electrode, a second electrode, and a pulse generator coupled to the first and second electrodes. The pulse generator is configured to apply a waveform between the first and second electrodes to perform irreversible electroporation, wherein the waveform includes pulses having a voltage amplitude of 1000 Volts (V) or less and an effective pulse width of 10 microseconds (μs) or more.

### CROSS REFERENCE TO RELATED APPLICATIONS

This application claims priority to U.S. Provisional Patent Application No. 63/562,504 filed on Mar. 7, 2024, which is incorporated by reference herein in its entirety.

### FIELD OF THE DISCLOSURE

The present disclosure relates generally to tissue ablation systems. In particular, the present disclosure relates to systems and methods for pulsed field ablation that use low voltage, long duration pulses.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to cause tissue destruction in cardiac tissue to correct conditions such as ventricular and atrial arrhythmias (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a substantially non-thermal ablation technique that involves applying strong electric fields that induce pore formation in the cellular membrane. The electric field may be induced by applying a relatively short duration pulse which may last, for instance, from a nanosecond to several milliseconds and generate a moderate amount of heating. Such a pulse may be repeated to form a pulse train. When such an electric field is applied to tissue in an in vivo setting, the cells in the tissue are subjected to an increased trans-membrane potential, which opens the pores on the cell plasma membrane. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

For example, pulsed field ablation (PFA) may be used to perform instantaneous pulmonary vein isolation (PVI). PFA generally involves delivering high voltage pulses from electrodes disposed on a catheter. For example, in at least some systems, voltage pulses may range from less than about 50 volts to about 10,000 volts or higher. These fields may be applied between pairs of electrodes (bipolar therapy) or between one or more electrodes and a return patch (monopolar therapy).

In PFA, different waveforms may be used to achieve different goals. For example, some waveforms may result in larger or smaller lesion size than other waveforms. Further, some waveforms result in higher or lower overall energy delivery than other waveforms (less overall energy delivery generally corresponds to less heating of the target tissue). As another example, some waveforms are more likely to induce muscular contractions in a patient. Generally, it is desirable to minimize thermal heating of the tissue, and to have little to no skeletal muscle recruitment (i.e., avoiding muscle contractions). In addition, it is also generally desirable to reduce the likelihood of waveforms generating sustained atrial arrhythmias.

During application of PFA, electrode geometry (e.g., electrode shape, length, interelectrode distances, etc.) determines the size of the electric field generated, and accordingly, the lesion size. In at least some known systems, PFA uses relatively short pulse durations and relatively high applied voltages to generate pores in the cell membrane to cause cell death. However, it would be desirable to achieve meaningful lesion formation at lower voltage levels than those used in at least some known systems.

### BRIEF SUMMARY OF THE DISCLOSURE

In one aspect, an electroporation system is provided. The system includes a catheter assembly including a first electrode, a second electrode, and a pulse generator coupled to the first and second electrodes. The pulse generator is configured to apply a waveform between the first and second electrodes to perform irreversible electroporation, wherein the waveform includes pulses having a voltage amplitude of 1000 Volts (V) or less and an effective pulse width of 10 microseconds (μs) or more.

In another aspect, a method for electroporation therapy is provided. The method includes generating, using a pulse generator, a waveform including pulses having a voltage amplitude of 1000 Volts (V) or less and an effective pulse width of 10 microseconds (μs) or more. The method further includes delivering, using a first electrode and a second electrode coupled to the pulse generator, the waveform between the first electrode and the second electrode to perform irreversible electroporation.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIGS. 2A and 2B are views of one embodiment of a catheter assembly that may be used with the system shown in FIG. 1.

FIGS. 3A-3C are views of alternative embodiments of a catheter assembly that may be used with the system shown in FIG. 1.

FIG. 4 is a schematic view of an example embodiment of a flexible printed electrode array.

FIG. 5 is a schematic diagram illustrating various tissue zones around an electrode during electrical pulse applications.

FIG. 6 illustrates a comparison of a first applied waveform and a second applied waveform.

FIG. 7 is a perspective view of one embodiment of a catheter assembly that may be used with the system shown in FIG. 1.

FIG. 8 is a perspective view of another embodiment of a catheter assembly that may be used with the system shown in FIG. 1.

FIG. 9 is a perspective view of another embodiment of a catheter assembly that may be used with the system shown in FIG. 1.

FIG. 10 is a perspective view of another embodiment of a catheter assembly that may be used with the system shown in FIG. 1.

FIG. 11 is a perspective view of another embodiment of a catheter assembly that may be used with the system shown in FIG. 1.

FIG. 12 is a perspective view of another embodiment of a catheter assembly that may be used with the system shown in FIG. 1.

### DETAILED DESCRIPTION OF THE DISCLOSURE

The present disclosure provides systems and methods for electroporation. An electroporation system includes a catheter assembly including a first electrode, a second electrode, and a pulse generator coupled to the first and second electrodes. The pulse generator is configured to apply a waveform between the first and second electrodes to perform irreversible electroporation, wherein the waveform includes pulses having a voltage amplitude of 1000 Volts (V) or less and an effective pulse width of 10 microseconds (μs) or more.

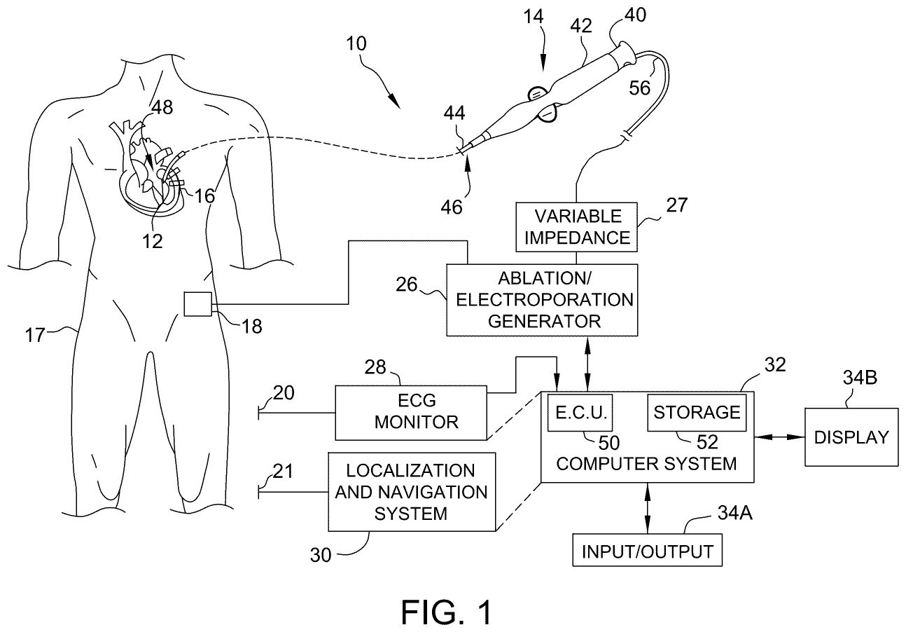

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

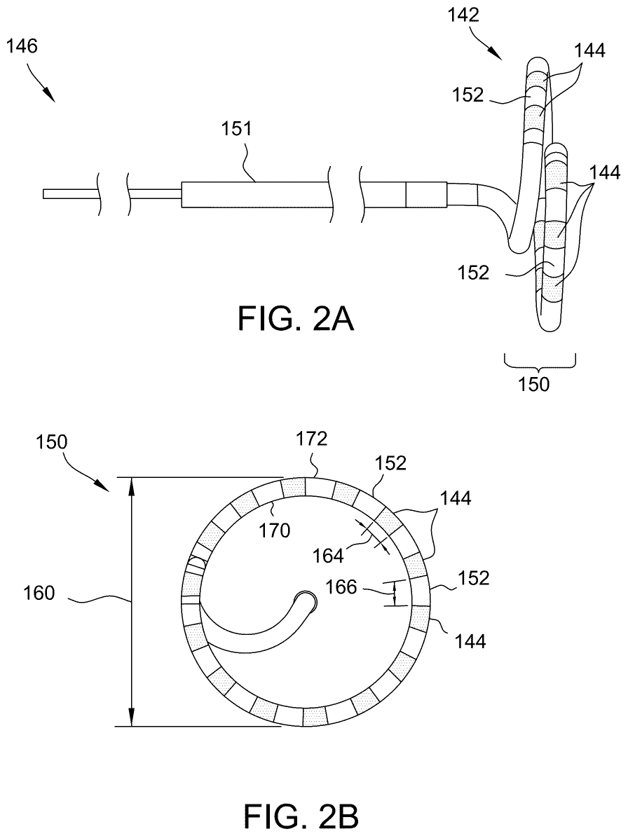

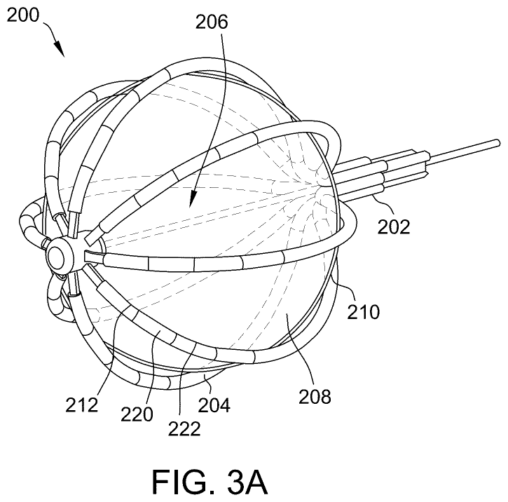

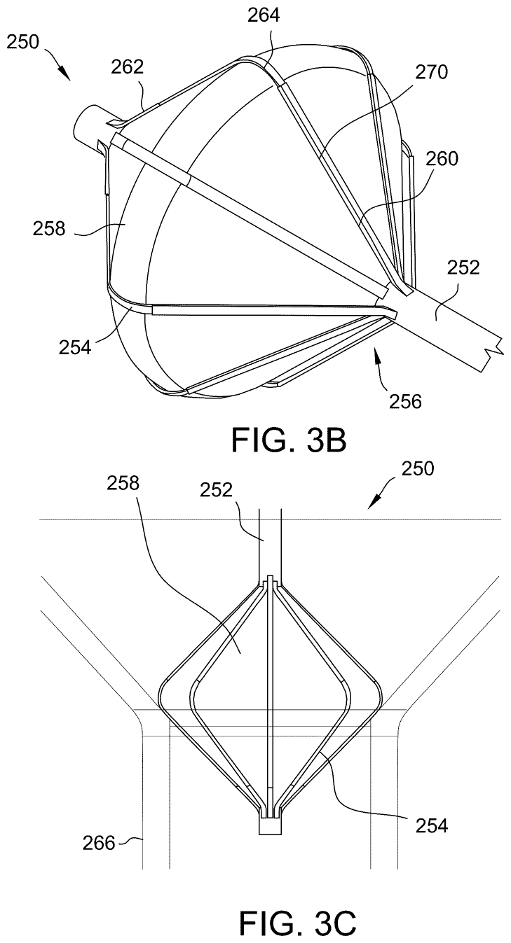

System 10 may be used for irreversible electroporation (IRE) to destroy tissue. In particular, system 10 may be used for electroporation-induced therapy that includes delivering electrical pulses in such a manner as to directly cause an irreversible loss of plasma membrane integrity leading to its breakdown and cell destruction. This mechanism of cell destruction may be viewed as an “outside-in” process, meaning that the disruption of the outside plasma membrane of the cell causes detrimental effects to the inside of the cell. Sometimes these electrical pulses may directly manipulate and damage the intracellular organelles to induce cell death, without causing a significant amount of damage to the cell membrane. Typically, for classical electroporation, electric energy may be delivered as a pulsed electric field in the form of short-duration pulses (e.g., having a 10 nanosecond (ns) to 100 millisecond (ms) duration) between closely spaced electrodes capable of delivering an electric field strength of about 0.05 to 100.0 kilovolts/centimeter (kV/cm). System 10 may be used for high output (e.g., high voltage and/or high current) electroporation procedures (and may also be used for low output electroporation procedures, as described herein). Further, system 10 may be used with a loop catheter such as that depicted in FIGS. 2A and 2B, and/or with a basket catheter such as those depicted in FIGS. 3A-3C. In some embodiments, system 10 is used for reversible electroporation instead of or in addition to irreversible electroporation.

Notably, in at least some of the embodiments described herein, system 10 operates at lower voltages than at least some known electroporation systems, as described herein.

In one embodiment, stimulation is delivered selectively (e.g., between pairs of electrodes) on catheter 14. Further, the electrodes on catheter 14 may be switchable between being connected to a 3D mapping system and being connected to an electroporation generator.

Irreversible electroporation through a multi-electrode catheter may enable pulmonary vein isolation in as few as one shock per vein, which may produce much shorter procedure times compared to sequentially positioning a radiofrequency (RF) ablation tip around a vein. Further, irreversible electroporation may be used for focal ablation procedures. Notably, the embodiments described herein may be used with any suitable irreversible electroporation application.

It should be understood that while the energization strategies are described as involving square wave pulses, embodiments may use variations and remain within the spirit and scope of the disclosure. For example, exponentially-decaying pulses, exponentially-increasing pulses, and combinations may be used.

Further, it should be understood that the mechanism of cell destruction in electroporation is not primarily due to heating effects, but rather to cell membrane disruption (e.g., through pore formation and/or other cell damage), damage to intracellular organelles and oxidate stress etc., causing a cell wide effect through application of a high-voltage electric field. Thus, electroporation may avoid some possible thermal effects that may occur when using radio frequency (RF) energy. This “colder therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues (e.g., renal tissue, tumors, etc.).

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). Further, in some embodiments, in a multiplexing arrangement, therapy may repeatedly switch between using a different return electrodes 18, 20, 21. In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation therapy, generator 26 may be configured to produce an electric energy that is delivered via electrode assembly 12 as a pulsed electric field in the form of short-duration square wave pulses (e.g., a nanosecond to several milliseconds duration, or any duration suitable for electroporation) between closely spaced electrodes capable of delivering an electric field strength (i.e., at the tissue site) of about 0.05 to 100.0 kV/cm. The amplitude and pulse width needed for irreversible electroporation are nearly inversely related. That is, as pulse widths are decreased, the amplitude may generally be increased to achieve chronaxie. The electric energy may be delivered, for example, using a fixed voltage delivery system (in which a fixed voltage is applied, independent of a patient impedance) or using a fixed current delivery system (in which a fixed current is achieved by adjusting the voltage based on the patient impedance). In a fixed current delivery system, the patient impedance may be determined, for example, by delivering a relatively small voltage pulse and measuring current to calculate impedance, or by delivering an AC current waveform and measuring voltage to calculate impedance. A fixed current system may also involve measuring current (e.g., before or during therapy delivery) and adjusting voltage accordingly. For example, current may be measured during delivery of a first therapy pulse (or during a pre-therapy pulse with a relatively low voltage), an impedance may be calculated from the measured current, and the voltage may be adjusted (and then left unchanged) to obtain the desired current during therapy. In another example, current may be measured during one or more pulses delivered during therapy, the impedance may be calculated for each pulse that the current was measured for, and the voltage of each subsequent pulse may be actively adjusted.

Electroporation generator 26, sometimes also referred to herein as a DC energy source, is a biphasic electroporation generator 26 configured to generate a series of energy pulses that all produce current in two directions (i.e., positive and negative pulses). In other embodiments, electroporation generator is a monophasic or polyphasic electroporation generator. In some embodiments, electroporation generator 26 is configured to output energy in pulses at selectable energy levels, such as fifty joules, one hundred joules, two hundred joules, and the like. Other embodiments may have more, or fewer energy settings and the values of the available setting may be the same or different (settings may include, e.g., waveform parameters, voltage, current, number of applications, etc.). For successful electroporation, some embodiments utilize the two hundred joule output level. For example, electroporation generator 26 may output a pulse having a peak magnitude from about 10 Volts (V) to about 20,000 V. Other embodiments may output any other suitable positive or negative voltage. As described in detail below, many of the embodiments described herein operate at relatively low voltage levels (as compared to at least some known electroporation systems).

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, biologics, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N.V., the AURORA® system of Northern Digital Inc., commonly available fluoroscopy systems, or a magnetic location system such as the gMPS system from Mediguide Ltd.

In this regard, some of the localization, navigation and/or visualization systems may include a sensor for producing signals indicative of catheter location information, and may include, for example, one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA), which is a methodology for achieving irreversible electroporation and cell death, may be implemented using the systems and methods described herein. In some cases, PFA may be used at specific cardiac tissue sites such as the pulmonary veins to perform a pulmonary vein isolation (PVI), or to perform focal ablation. In PFA, electric fields may be applied between adjacent electrodes (in a bipolar approach) or between one or more catheter electrodes and a return patch (in a monopolar approach) or a combination of catheter electrodes and a return patch (in a multipolar approach). There are advantages and disadvantages to each of these approaches.

Both approaches, using an appropriate electrode geometry, are able to provide contiguous lesions. For lesion size and proximity, the monopolar approach can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions (depending on, for example, tissue thickness). To monitor operation of system 10, one or more impedances between catheter electrodes 144 and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and U.S. Patent Application No. 63/027,660, filed on May 20, 2020, all of which are incorporated by reference herein in their entirety.

FIGS. 2A and 2B are views of one embodiment of a catheter assembly 146 that may be used with catheter 14 in system 10. Catheter assembly 146 may be referred to as a loop catheter.

Those of skill in the art will appreciate that, in other embodiments, any suitable catheter may be used. That is, the systems and methods described herein are not limited to use with the particular catheter assemblies shown. For example, the systems and methods described herein may be implemented in a linear catheter, a grid catheter (e.g., a catheter including a number of splines arranged in a plane, each spline including one or more electrodes), and/or a focal ablation catheter (e.g., such as the Abbott TactiFlex catheter and TactiCath catheter).

Specifically, FIG. 2A is a side view of catheter assembly 146 with a variable diameter loop 150 at a distal end 142. FIG. 2B is an end view of variable diameter loop 150 of catheter assembly 146. Those of skill in the art will appreciate that the methods and systems described herein may be implemented using any suitable catheter (e.g., fixed loop catheters, linear catheters, basket catheter, etc.). As shown in FIGS. 2A and 2B, variable diameter loop 150 is coupled to a distal section 151 of shaft 44.

Variable diameter loop 150 is selectively transitionable between an expanded (also referred to as “open”) diameter 160 (shown in FIG. 2A) and a retracted (also referred to as “closed”) diameter 160 (not shown). In the example embodiment, an expanded diameter 160 is twenty eight mm and a retracted diameter 160 is fifteen mm. In other embodiments, diameter 160 may be variable between any suitable open and closed diameters 160.

In the embodiment shown, variable diameter loop 150 includes fourteen catheter electrodes 144 substantially evenly spaced around the circumference of variable diameter loop 150 in the expanded configuration. In the retracted configuration, one or more of electrodes 144 may overlap. In other embodiments, other arrangements of catheter electrodes 144 may be implemented. For example, in one embodiment, variable diameter loop 150 includes twelve catheter electrodes 144.

Catheter electrodes 144 are platinum ring electrodes (or electrodes made of other suitable materials) configured to conduct and/or discharge electrical current at any suitable voltage and/or current. For example, as described herein, at least some of the embodiments of the disclosure operate at voltage levels lower than those of at least some known electroporation systems. In other embodiments, variable diameter loop 150 may include any suitable number of catheter electrodes 144 made of any suitable material. Catheter electrodes 144 may include any catheter electrode suitable to conduct voltages and currents as described herein. Each catheter electrode 144 is separated from each other catheter electrode by an insulated gap 152. In the example embodiment, each catheter electrode 144 has a same length 164 (shown in FIG. 2B) and each insulated gap 152 has a same length 166 as each other gap 152. Length 164 and length 166 are both about 2.5 mm in the example embodiment. In other embodiments, length 164 and length 166 may be different from each other. Moreover, in some embodiments, catheter electrodes 144 may not all have the same length 164 and/or insulated gaps 152 may not all have the same length 166. In some embodiments, catheter electrodes 144 are not spaced evenly around the circumference of variable diameter loop 150.

Diameter 160 and catheter electrode 144 spacing may be developed to provide a targeted range of energy density to tissue, as well as to provide sufficient electroporation coverage for different human anatomic geometries. In general, a sufficient number of electrodes 144 with appropriate lengths 164 are desired to provide substantially even and continuous coverage around the circumference of variable diameter loop 150, while still allowing enough flexibility to allow variable diameter loop 150 to expand and contract to vary diameter 160 to the desired extremes.

As mentioned above, length 164 of catheter electrodes 144 may be varied. Increasing length 164 of catheter electrodes 144 may increase coverage of electrodes 144 around the circumference of variable diameter loop 150 while also decreasing current density (by increasing the surface area) on electrodes 144, which may help prevent arcing and reduce thermal effects during electroporation operations. Increasing length 164 too much, however, may prevent variable diameter loop 150 from forming a smooth circular shape and may limit the closed diameter 160 of variable diameter loop 150. Additionally, too great a length 164 may increase the surface area of catheter electrodes 144 to a point that the current density applied to catheter electrodes 144 by a power source is below the minimum current density needed for successful therapy. Conversely, decreasing length 164 decreases the surface area, thereby increasing the current density (assuming no other system changes) on catheter electrodes 144. As discussed above, greater current densities may lead to increased risk of arcing and heating during electroporation, and may result in larger additional system resistances needing to be added to prevent arcing. Moreover, in order to get a desired, even coverage about the circumference of variable diameter loop 150, more catheter electrodes 144 may be needed if length 164 is decreased. Increasing the number of catheter electrodes 144 on variable diameter loop 150 may prevent variable diameter loop 150 from being able to be contracted to a desired minimum diameter 160.

FIG. 3A is a perspective view of an alternative catheter assembly 200 that may be used with catheter 14. Catheter assembly 200 may be referred to as a basket catheter. Catheter assembly 200 includes a shaft 202 and a plurality of splines 204 surrounding a distal portion 206 of shaft 202. In this embodiment, catheter assembly 200 also includes a balloon 208 enclosed by splines 204. Balloon 208 may be selectively inflated to fill the space between splines 204. Notably, balloon 208 functions as an insulator, and generally reduces energy losses, which may result in increased lesion size. In some embodiments, balloon 208 may be filled with a cold media (e.g., a cryofluid or cold saline). Further, in some embodiments balloon 208 may be a double-layered balloon.

Each spline 204 includes a proximal end 210 coupled to shaft 202 and a distal end 212 coupled to shaft 202. From proximal end 210 to distal end 212, spline 204 has an arcuate shape that extends radially outward.

In this embodiment, each spline 204 includes a plurality of individual electrodes 220. For example, each spline 204 may include an elastic material (e.g., Nitinol) covered in a polymer tube 222, with individual electrodes 220 attached to an exterior of polymer tube 222. In the embodiment shown, each spline 204 includes two electrodes 220. Further, as shown in FIG. 2, electrodes 220 are generally positioned closer to distal end 212 than proximal end 210 to correspond to portions of spline 204 that will contact the pulmonary vein.

Alternatively, each spline 204 may include any suitable number and arrangement of electrodes 220. For example, in some embodiments, each spline 204 includes four electrodes 220.

In this embodiment, alternating splines 204 alternate polarities. That is, electrodes 220 on a particular spline 204 have the same polarity, but electrodes 220 on a particular spline 204 have a different polarity than electrodes 220 on adjacent splines 204. Alternatively, any suitable polarization scheme may be used. During delivery, splines 204 may be collapsed in towards shaft 202. Subsequently, to perform ablation, splines 204 are deployed to extend radially outward.

Splines 204 may all have the same length, or at least some of splines 204 may have different lengths. Further, insulating material on each spline 204 may have the same length, or at least some splines 204 may have insulating material with different lengths. In addition, in some embodiments, catheter assembly 200 includes a distal electrode (not shown) positioned distal of splines 204. The distal electrode may be used to perform point ablation (e.g., by creating a bipole between the distal electrode and one of splines 204), and/or may be used for visualization/mapping purposes (e.g., using the distal electrode in combination with an electrode on shaft 202).

FIG. 3B is a perspective view of an alternative catheter assembly 250 that may be used with catheter 14, and FIG. 3C is a side schematic view of catheter assembly 250. Like catheter assembly 200 (shown in FIG. 3A), catheter assembly 250 may be referred to as a basket assembly.

Catheter assembly 250 includes a shaft 252 and a plurality of splines 254 surrounding a distal portion 256 of shaft 252. In this embodiment, catheter assembly 250 includes a balloon 258 enclosed by splines 254. Balloon 258 may be selectively inflated to occupy the space between splines 254. Notably, balloon 258 functions as an insulator, and generally reduces energy, which may result in increased lesion size.

Each spline 254 includes a proximal end 260 coupled to shaft 252 and a distal end 262 coupled to shaft 252. From proximal end 260, spline 1004 extends radially outward to an inflection point 264, and then extends radially inward to distal end 262. FIG. 3C shows catheter assembly 250 positioned within the pulmonary vein 266.

A body of each spline 254 is made of an elastic material (e.g., Nitinol), and functions as a relatively large electrode. In this embodiment, alternating splines 254 alternate polarities. That is, each positive spline 254 is positioned between two negative splines 254 and vice-versa. Alternatively, any suitable polarization scheme may be used.

To control the ablation zone of each spline 254, portions of each spline 254 may be covered with insulating material 270 (e.g., heat-shrink or polymer tubing or spray or dip coat with polyimide or PEBAX), and the exposed portions of splines 254 function as electrodes. In the embodiment shown in FIGS. 3B and 3C, inflection point 264 and portions of spline 254 between inflection point 264 and distal end 262 are generally exposed, while portions of spline 254 between inflection point 264 and proximal end 260 are generally insulated. This results in the portions of spline 254 that contact pulmonary vein 266 being exposed (see FIG. 3C). Alternatively, any suitable insulation configuration may be used.

During delivery, splines 254 and balloon 258 may be collapsed. To perform ablation, splines 254 are deployed with inflection points 264 extending radially outward, and balloon 258 is selectively inflated to occupy the space between splines 254.

The combination of balloon 258 and splines 254 facilitates straightforward delivery and deployment of catheter assembly 250. Further, balloon 258 drives more energy into ablated tissue, and stabilizes splines 254 to prevent lateral movement. In addition, using splines 254 as electrodes instead of individual smaller electrodes may facilitate reducing the cost and increasing the reliability of catheter assembly 250.

Splines 254 may all have the same length, or at least some of splines 254 may have different lengths. Further, insulating material 270 on each spline 254 may have the same length, or at least some splines 254 may have insulating material 270 with different lengths. In addition, in some embodiments, catheter assembly 250 includes a distal electrode (not shown) positioned distal of splines 254. The distal electrode may be used to perform point ablation (e.g., by creating a bipole between the distal electrode and one of splines 254), and/or may be used for visualization/mapping purposes (e.g., using the distal electrode in combination with an electrode on shaft 252).

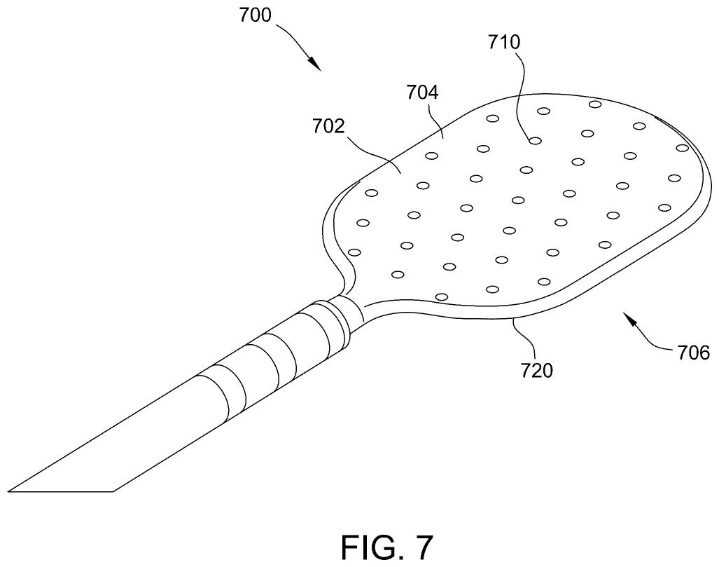

Those of skill the art will appreciate that catheter assembly 146 (shown in FIGS. 2A and 2B), catheter assembly 200 (shown in FIG. 3A), and catheter assembly 250 (shown in FIGS. 3B and 3C) are merely examples. Notably, the systems and methods described herein may be implemented using any suitable catheter assembly. For example additional catheter assemblies are described below in relation to FIGS. 7-10.

For electroporation therapy, waveforms are generated using a pulse generator (e.g., electroporation generator 26 (shown in FIG. 1)) and applied between two or more catheter electrodes (i.e., a bipolar approach) or between individual catheter electrodes and a return patch (i.e., a monopolar approach). The waveforms may be monophasic, biphasic (i.e., having both a positive pulse and a negative pulse), or polyphasic. Further, the waveforms may include one or more bursts of pulses (with each burst including multiple pulses). Further, the waveforms are defined by multiple parameters (e.g., pulse width, pulse amplitude, frequency, etc.).

Different waveforms may be used to achieve different goals. For example, some waveforms may result in larger or smaller lesion size than other waveforms. Further, some waveforms result or higher or lower overall energy delivery than other waveforms (less overall energy delivery generally corresponds to less heating of the target tissue). As another example, some waveforms are more likely to induce muscular contractions in a patient. It is generally desirable to avoid thermal heating of the tissue, and to have little to no skeletal muscle recruitment (i.e., avoiding muscle contractions).

In at least some known systems, waveforms with relatively short duration pulses and relatively high voltage pulses are used to cause cell death. However, as described herein, effective lesion formation may still be maintained while using lower voltage pulses.

Notably, when electrodes are positioned relatively close to one another, a relatively high electric field strength may be obtained, even at relatively low applied voltages. Relatively close spacing of electrodes may be achieved using flexible printed electrodes, as described herein. Using such a configuration, lesions may be generated at relatively low applied voltages. Those lesions may be shallower, as the electric field generally decreases relatively quickly away from the closely positioned electrodes. However, as described herein, longer duration pulses may be helpful in decreasing the electric field threshold required for irreversible electroporation, thus increasing ablation area while still taking advantage of relatively low applied voltages. As used herein, relatively low applied voltages may refer to an applied voltage less than 1000 Volts, more particularly an applied voltage less than 500 Volts, even more particularly an applied voltage less than 200 Volts, and even more particularly an applied voltage less than 50 Volts.

The embodiments described herein provide strategies for relatively low voltage PFA using relatively long duration pulses between electrodes placed relatively close to one another (such as flexible printed electrodes). These arrangements facilitate reducing complexities of catheter and generator designs, and facilitate reducing collateral responses (e.g., muscle stimulation, cough response, vasal spasm, nerve damage, etc.) due to smaller penetration of relatively high intensity electric fields. The electrodes may include electrodes on the same catheter, electrodes on separate catheters, and/or an electrode on a catheter and an external patch electrode.

The system and methods described herein enable lesion formation at relatively low applied voltages by using two flexible printed electrodes placed relative close to one another. Applied pulses may have a pulse width (or effective pulse width) of at least 10 microseconds (μs) in some embodiments, and at least 100 μs in some embodiments. Further, as described herein, relatively long pulse widths (e.g., on the order of 10 μs (or 100 μs) to 20 milliseconds (ms)) may be effectively achieved using multiple pulses with relatively short pulse widths (e.g., on the order of 10 nanoseconds (ns) to 10 μs) that are closely spaced together (e.g., with consecutive pulses separated by a relatively short time gap on the order of 10 ns to 1 ms). Accordingly, a continuous energy application is not required to achieve the benefits of longer pulse widths, and the advantages offered by relatively short pulse widths (e.g., reduced muscle stimulation) may still be realized.

In at least some known PFA systems, applied pulses have relatively large voltages (e.g., on the order of hundreds to thousands of volts) at relatively high currents (e.g., on the orders of tens of amps). These electrical loads are generally above the performance achievable using flexible printed circuits. For example, flexible printed circuits may not be able to carry relatively high voltage electrical loads, and using relatively high currents may damage various materials in flexible printed circuits. However, by using lower voltage pulses, as described herein, flexible printed circuits may be employed.

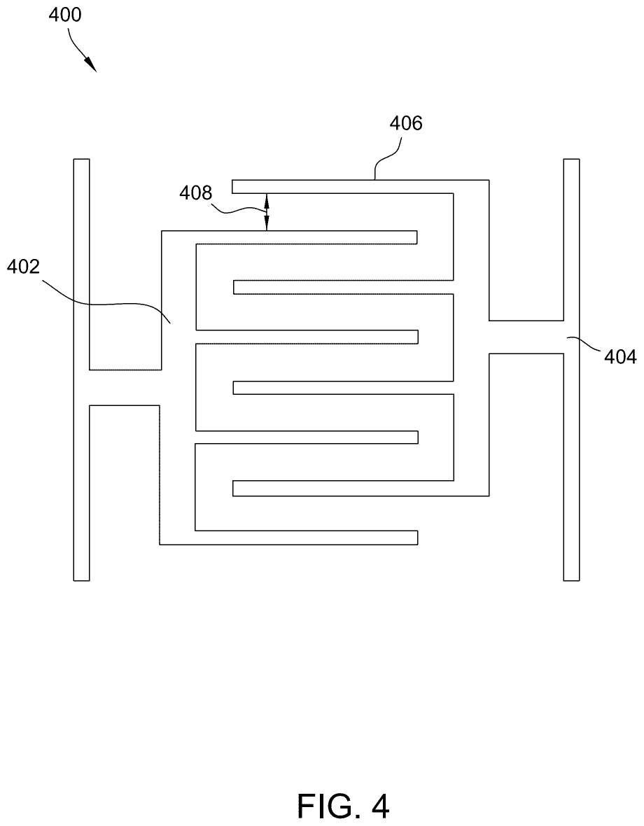

FIG. 4 is a schematic view of an example embodiment of a flexible printed electrode array 400. In this embodiment, array 400 includes a first electrode 402 and a second electrode 404. Electrodes 402 and 404 each include a plurality of fingers 406 that form an interlocking pattern, with a gap 408 defined between each finger 406 of first electrode 402 and an adjacent finger 406 of second electrode 404.

Gap 408 constitutes a relatively small intra-electrode distance that facilitates generating relatively high intensity electric fields at relatively low applied voltages. Gap 408 may be, for example, 2,000 micrometers (μm) or less, more particularly 1,000 μm or less, more particularly 500 μm or less, or more particularly 50 μm or less.

Those of skill in the art will appreciate that these values are merely examples, and gap 408 may have any suitable dimensions. Further, the flexible printed electrode arrays used in the embodiments disclosed herein may have a different arrangement than that shown in FIG. 4 (i.e., different from a plurality of interlocking fingers). For example, in some embodiments, a flexible printed electrode array is implemented using a substantially transparent device that includes poly(3,4-ethylenedioxythiophene) polystyrene sulfonate (PEDOT:PSS).

As noted above, the relatively small gap 408 enables generating relatively high intensity electric fields at relatively low applied voltages. For example, the following Table 1 lists applied voltages necessary to achieve a target electric field intensity at a given gap 408 dimension.

TABLE 1

| Target | Electrode | | | | |
|---|---|---|---|---|---|
| E-field | Gap Distance | | | | |
| (V/cm) | (μm)→ | 2,000 | 1,000 | 500 | 50 |
| 100 | Applied Voltage (V) | 20 | 10 | 5 | 0.5 |
| | for target E-field → | | | | |
| 200 | Applied Voltage (V) | 40 | 20 | 10 | 1 |
| | for target E-field → | | | | |
| 400 | Applied Voltage (V) | 80 | 40 | 20 | 2 |
| | for target E-field → | | | | |
| 1,000 | Applied Voltage (V) | 200 | 100 | 50 | 5 |
| | for target E-field → | | | | |

As can be seen from Table 1, the shorter the gap dimension, the less applied voltage needed to generate a given electric field. These electric fields are sufficient to cause effective ablation in areas where the electric field exceeds the IRE threshold. The IRE threshold itself is dictated by waveform parameters (e.g., pulse width, number of pulses, pulse amplitude, repetition rate). Notably, despite the generation of relatively high electric fields by closely spaced electrodes, the tissue depth of the electric field tends to be relatively limited, and fall off relatively quickly, resulting in shallow lesions. To compensate for this, the applied voltage may be increased. However, this may result in increased electrolytic and/or thermal damage.

Accordingly, in the methods and systems described herein, instead of increasing the applied voltage, longer pulse widths (e.g., on the order of 10 μs (or 100 μs) to 20 ms) may be used to increase lesion depth when using flexible printed electrodes at relatively low applied voltages. For example, suitable pulse widths may include pulse widths greater than or equal to 10 μs more particularly greater than or equal to 100 μs, more particularly greater than or equal to 500 μs, more particularly greater than or equal to 1 ms, more particularly greater than or equal to 10 ms, or more particularly greater than or equal to 20 ms.

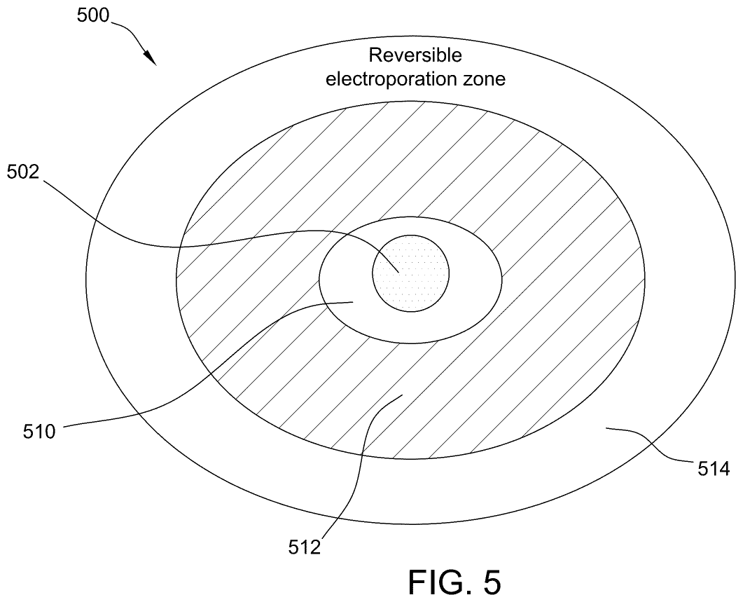

FIG. 5 is a schematic diagram 500 illustrating various tissue zones around an electrode 502 during electrical pulse applications delivered using electrode 502. For illustration purposes, only one electrode 502 is shown. As shown in FIG. 5, tissue zones includes an inner zone 510, an intermediate zone 512, and an outer zone 514. Inner zone 510 represents a zone in which tissue is subject to IRE, as well as heating and electrolysis. Intermediate zone 512 represents a zone in which tissue is subject to IRE (but not subject to heating and electrolysis). Finally, outer zone 514 represents a zone in which tissue is subject to reversible electroporation. The boundaries of zones 510, 512, and 514 are governed by the distribution of the generated electric field, IRE threshold, electrode geometry, and heating and electrolysis generated by a particular waveform. As noted above, waveform parameters dictate the IRE threshold.

Notably, for longer duration pulses, the IRE threshold is lower than for shorter duration pulses. Accordingly, longer duration pulses increase the IRE zone (i.e., intermediate zone 510) and push the reversible electroporation zone (i.e., outer zone 514) further out. This results in improved lesion depth for the same applied voltage, as lesions are generated further from the electrodes (than when using shorter duration pulses).

Using longer pulses facilitates generating deeper lesions when using flexible printed electrodes, where the reach of a relatively high intensity electric field is relatively shallow.

Notably, a continuous application of relatively long pulses may increase the potential for muscle stimulation, which may lead to patient discomfort. Further, continuous application of relatively low voltage pulses may also increase the amount of electrolysis, leading to microbubble production and tissue heating.

Accordingly, in at least some embodiments, as noted above, instead of applying a long continuous pulse, multiple pulses with relatively short pulse widths (e.g., on the order of 10 nanoseconds (ns) to 10 μs) are closely spaced together (e.g., with consecutive pulses separated by a relatively small time gap on the order of 10 ns to 1 ms) to effectively achieve relatively long pulse widths.

Electroporation is considered a membrane phenomenon along with cell wide effects produced by that phenomenon. When cells are tissues are subjected to relatively short duration, high voltage electrical pulses, a transmembrane voltage increases. When the transmembrane voltage increases beyond a threshold (e.g., 0.5 V), across a relatively thin membrane (e.g., approximately 5 nanometers (nm) in thickness), there is a several-fold enhancement of the electric field (e.g., 100 megavolts/centimeter), leading to pore formation. In order to cause electroporation, the transmembrane voltage must stay above the threshold for a certain amount of time.

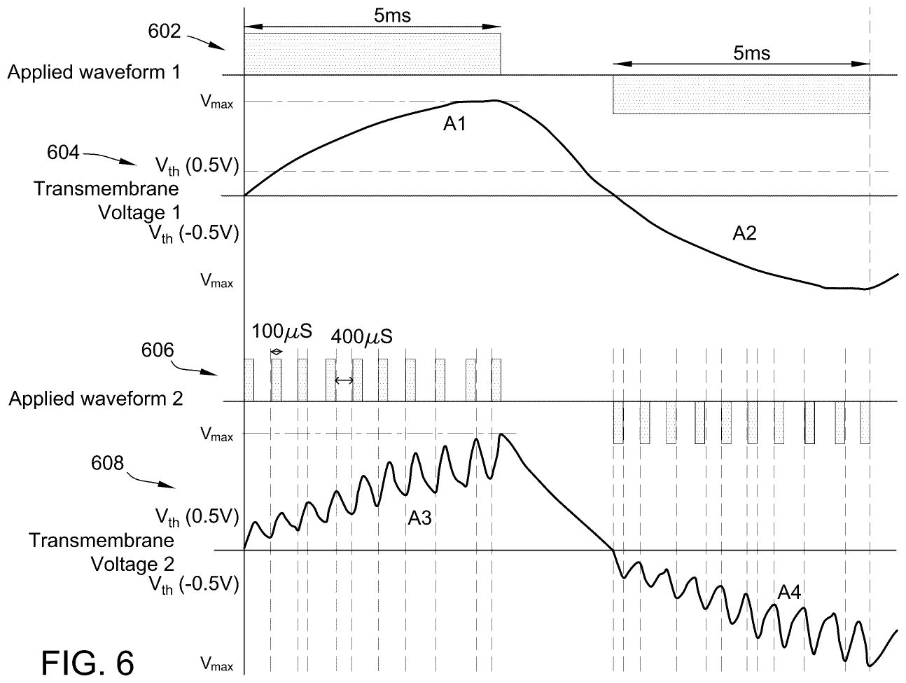

Longer pulse widths, higher applied voltages, and increased numbers of pulses increase the time above the threshold to cause more effective electroporation. Notably, however, in the embodiments described herein, the applied voltage need not be continuously applied to achieve electroporation. Rather, due to the capacitive nature of the cell membrane, an increase in the transmembrane voltage may be achieved by applying multiple closely spaced smaller duration pulses. FIG. 6 demonstrates this concept.

Specifically, FIG. 6 includes a first applied waveform 602 including a 5 ms positive pulse followed by a 5 ms negative pulse. First applied waveform 602 results in first transmembrane potential 604. When applying the relatively long 5 ms positive pulse, the transmembrane potential (or transmembrane voltage) increases steadily and is saturated at a highest possible transmembrane potential (Vₘₐₓ). Upon removal of the positive pulse, the transmembrane voltage decays slowly following an RC time constant of the membrane due to the capacitive nature of the membrane. A similar phenomenon occurs with the 5 ms negative pulse is applied.

In contrast, a second applied waveform 606 includes a plurality of smaller closely spaced positive pulses and closely spaced negative pulses (e.g., 100 μs pulses with a 400 μs delay between pulses), which results in second transmembrane potential 608.

As shown in FIG. 6, the small pulses of second applied waveform 606 result in second transmembrane potential 608 having a sawtooth-like shape. However, second transmembrane potential 608 still maintains a similar area under the curve as first transmembrane potential 604, which is sufficient for effective electroporation. That is the area under first transmembrane potential 604 (A1+A2) is substantially equal to the area under second transmembrane potential 608 (A3+A4).

In second applied waveform 606, the membrane potential discharges naturally during the gaps between pulses. This allows the membrane potential to stay above the electroporation threshold without requiring constant energy delivery. This concept could be extended to tens or hundreds of nanosecond pulses with optimized gaps between pulses to generate effective electroporation. Depending upon the gaps between pulses, it is possible to optimize an off time that achieves a maximum area above the electroporation threshold while minimizing a number of pulses. The optimized off time would also depend on the pulse width. For example, a nanosecond pulse (as compared to a microsecond pulse) will charge the membrane less and have a faster discharge, therefore requiring a smaller off time.

Using this concept, tens to hundreds of short (e.g., 10 ns to 100 μs, more particularly 10 ns to 10 μs, more particularly 10 ns to 1 μs, or even more particularly 10 ns to 500 ns) pulses may be applied with an optimized delay between pules to generate effective electroporation. In contrast to using relatively high applied voltages for nanosecond pulses to achieve high electric field thresholds for electroporation, this implementation takes advantage of sawtooth stacking of the transmembrane voltage using lower magnitude pulses and lower electric field strength using nanosecond pulses. The nanosecond pulses may facilitate reduced muscle stimulation, reduced electrolysis, while directly manipulating intracellular organelles (e.g., mitochondria, endoplasmic reticulum, etc.) to induce effective cell death. As cardiomyocytes appear to have more mitochondria as compared to other issues types, this treatment is particularly effective for cardiomyocytes.

In some embodiments, multiple different pulse widths could be combined. For example, an initial longer pulse (e.g., 1-10 μs in length) could be applied to induce an initially high transmembrane voltage, followed by a series of multiple smaller nanosecond pulses with delays to induce a sawtooth-shaped transmembrane potential, thereby causing effective ablation. Such a strategy further reduces the electric field required for electroporation, as the majority of the energy needed for initial pore formation will be achieved from the initial pulse, and the subsequent smaller pulses will hold the pores open, while manipulating the intracellular organelles to cause cell death. By using multiple smaller pules, instead of a larger continuous pulse, applied energy is reduced, thermal damage potential is limited, and potential for microbubbles and arrhythmia is also reduced.

Those of skill in the art will appreciate that any suitable catheter assembly including relatively closely spaced electrodes may be used to implement the embodiments described herein.

For example, FIG. 7 is a perspective view of one embodiment of a catheter assembly 700 that may be used with the embodiments described herein. Catheter assembly 700 may be referred to as a paddle catheter.

Catheter assembly 700 includes a relatively flat body 702 having a first side 704 and an opposite second side 706. Each side 704 and 706 includes an array of relatively closely spaced electrodes 710. In one embodiment, electrodes 710 on first side 704 are set to a first polarity, and electrodes 710 on second side 706 are set to a second, opposite polarity. In some embodiments, catheter assembly 700 further includes at least one elongated electrode 720 that extends around at least a portion of a perimeter of body 702. Electrodes 710 (and elongated electrode 720 in certain embodiments) are spaced relatively close to one another, facilitating generating lesions using relatively low applied voltages at relatively long pulse durations, as described above.

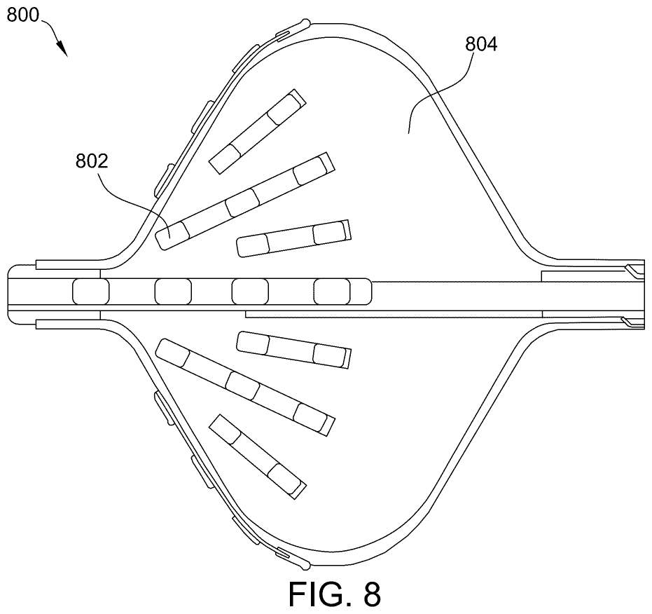

FIG. 8 is another embodiment of a catheter assembly 800 that may be used with the embodiments described herein. Catheter assembly 800 may be referred to as a balloon catheter. As shown in FIG. 8, catheter assembly 800 includes a plurality of electrodes 802 (e.g., flexible printed electrodes) located on a selectively inflatable balloon 804. Electrodes 802 are spaced relatively close to one another, facilitating generating lesions using relatively low applied voltages at relatively long pulse durations, as described above.

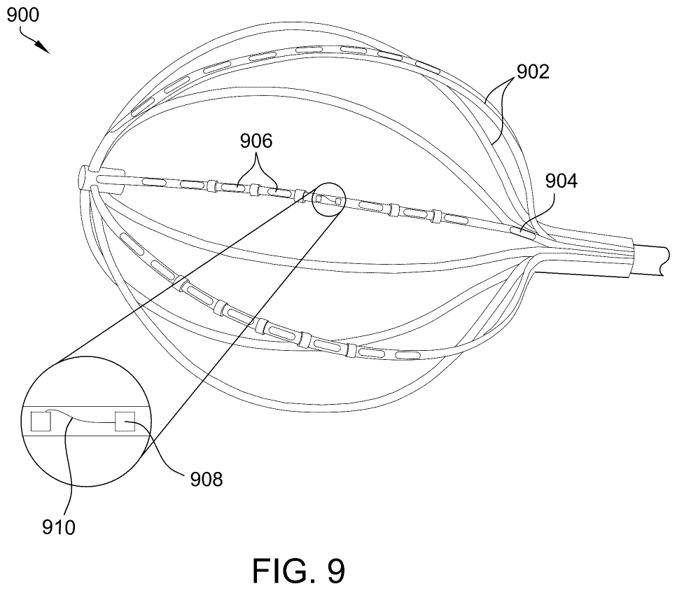

FIG. 9 is another embodiment of a catheter assembly 900 that may be used with the embodiments described herein. Catheter assembly 900 may be referred to as a basket catheter. As shown in FIG. 9, catheter assembly 900 includes a plurality of selectively expandable splines 902. At least some of splines 902 include one or more electrodes 904. The electrodes 904 may include elongated electrodes 906 and/or spot electrodes 908 (e.g., including flexible printed electrodes). One or more of spot electrodes 908 may be electrically coupled to one another using conductive traces 910. Here, electrodes 904 are spaced relatively close to one another, facilitating generating lesions using relatively low applied voltages at relatively long pulse durations, as described above.

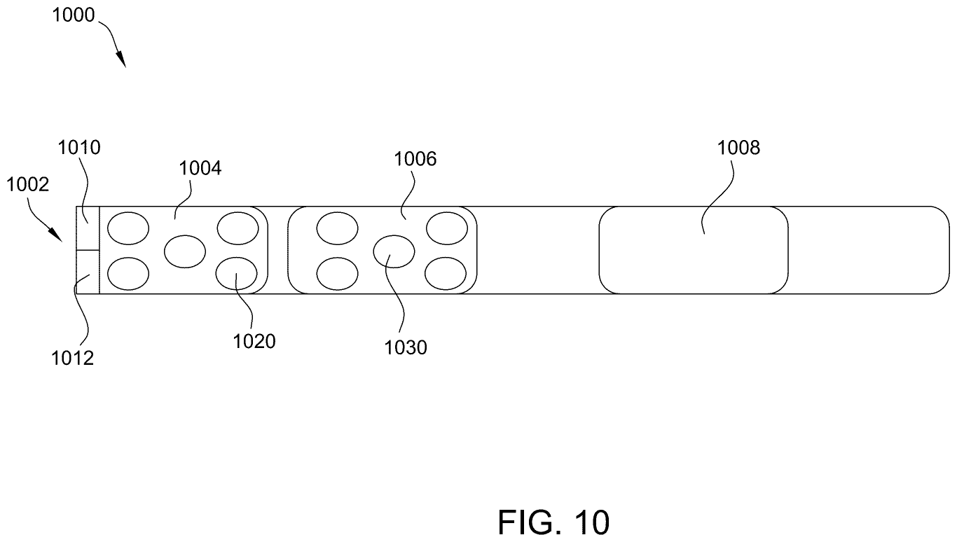

FIG. 10 is a schematic diagram of another embodiment of a catheter assembly 1000 that may be used with the embodiments described herein. Catheter assembly 1000 may be referred to as a linear catheter. Catheter assembly 1000 includes a tip electrode array 1002, a first electrode array 1004, a second electrode array 1006, and a ring electrode 1008. Tip electrode array 1002 includes a first tip electrode 1010 and a second tip electrode 1012. First electrode array 1004 includes a plurality of electrodes 1020 (e.g., flexible printed electrodes), and second electrode array 1006 includes a plurality of electrodes 1030 (e.g., flexible printed electrodes). Electrodes 1020 and 1030 are spaced relatively close to one another, facilitating generating lesions using relatively low applied voltages at relatively long pulse durations, as described above.

Those of skill in the art will appreciate that catheter assembly 1000 includes an example configuration of electrodes, and that other suitable arrangements of electrodes are possible on a linear catheter assembly (as well as on the other types of catheter assemblies discussed herein). For example, in some embodiments, flexible printed electrodes are arranged on a linear catheter assembly in a grid array (e.g., with sets of four electrodes forming a square or rectangular pattern). In other embodiments, flexible printed electrodes are rectangular, and arranged in a brick-like pattern on a linear catheter assembly. In other embodiments, flexible printed electrodes are arranged on a catheter assembly having an expandable form (e.g., an expandable basket).

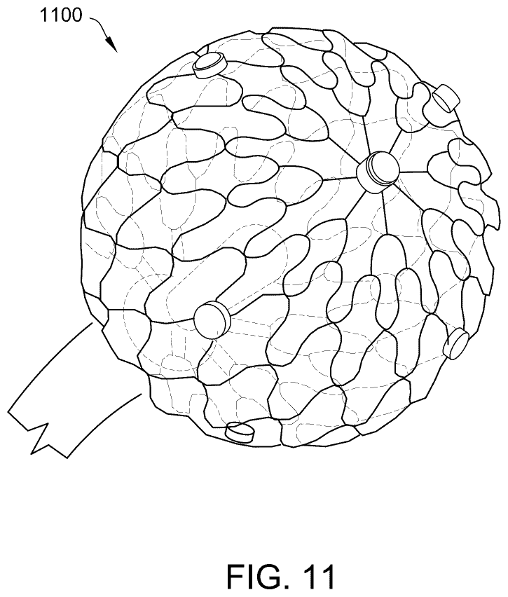

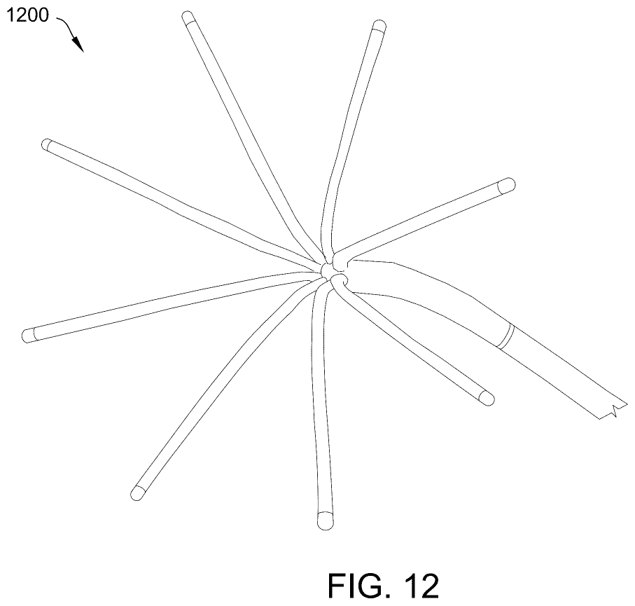

As another example, the systems and methods described herein may be implemented on a catheter assembly that includes electrodes arranged on and/or within a basket formed from a lattice of structs (see, e.g., catheter assembly 1100 shown in FIG. 11). As yet another example, the systems and methods described herein may be implemented on a catheter assembly that includes electrodes arranged on multiple cantilevered struts extending outward from a catheter shaft (see, e.g., catheter assembly 1200 shown in FIG. 12).

The embodiments and techniques described herein provide strategies for relatively low voltage PFA using relatively long duration pulses between electrodes placed relatively close to one another (such as flexible printed electrodes). These arrangements facilitate reducing complexities of catheter and generator designs, and facilitate with reduced collateral responses (e.g., muscle stimulation, cough response, vasal spasm, nerve damage, etc.) due to smaller penetration of relatively high intensity electric fields.

The systems and methods described herein are directed electroporation applications. An electroporation system includes a catheter assembly including a first electrode, a second electrode, and a pulse generator coupled to the first and second electrodes. The pulse generator is configured to apply a waveform between the first and second electrodes to perform irreversible electroporation, wherein the waveform includes pulses having a voltage amplitude of 1000 Volts (V) or less and an effective pulse width of 10 microseconds (μs) or more.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An electroporation system comprising:
a catheter assembly comprising a first electrode;
a second electrode; and
a pulse generator coupled to the first and second electrodes, the pulse generator configured to:
apply a waveform between the first and second electrodes to perform irreversible electroporation, wherein the waveform includes pulses having a voltage amplitude of 1000 Volts (V) or less and an effective pulse width of 10 microseconds (μs) or more.

2. The electroporation system in accordance with claim 1, wherein the first and second electrodes are separated by a distance of 2,000 micrometers (μm) or less.

3. The electroporation system in accordance with claim 1, wherein the first and second electrodes comprise flexible printed electrodes.

4. The electroporation system in accordance with claim 1, wherein the effective pulse width is achieved using a single continuous pulse.

5. The electroporation system in accordance with claim 1, wherein the effective pulse width is achieved using a plurality of pulses each having a pulse width of 10 μs or less and separated from one another by a time gap of 10 nanoseconds (ns) or more.

6. The electroporation system in accordance with claim 1, wherein the effective pulse width is achieved using an initial pulse having a pulse width of 1 μs or more followed by a plurality of pulses having a pulse width of less than 1 μs.

7. The electroporation system in accordance with claim 1, wherein the pulses of the waveform have an amplitude of 50V or less.

8. The electroporation system in accordance with claim 1, wherein the pulses have an effective pulse width of 500 μs or more.

9. The electroporation system in accordance with claim 1, wherein the catheter assembly comprises a basket catheter.

10. The electroporation system in accordance with claim 1, wherein the catheter assembly comprises a balloon catheter.

11. The electroporation system in accordance with claim 1, wherein the catheter assembly comprises a paddle catheter or a linear catheter.

12. The electroporation system in accordance with claim 1, wherein the second electrode is located on the catheter assembly.

13. A method for electroporation therapy, the method comprising:
generating, using a pulse generator, a waveform including pulses having a voltage amplitude of 1000 Volts (V) or less and an effective pulse width of 10 microseconds (μs) or more; and
delivering, using a first electrode and a second electrode coupled to the pulse generator, the waveform between the first electrode and the second electrode to perform irreversible electroporation.

14. The method in accordance with claim 13, wherein the first and second electrodes are separated by a distance of 2,000 micrometers (μm) or less.

15. The method in accordance with claim 13, wherein the first and second electrodes are flexible printed electrodes.

16. The method in accordance with claim 13, wherein the effective pulse width is achieved using a single continuous pulse.

17. The method in accordance with claim 13, wherein the effective pulse width is achieved using a plurality of pulses each having a pulse width of 10 μs or less and separated from one another by a time gap of 10 nanoseconds (ns) or more.

18. The method in accordance with claim 13, wherein the effective pulse width is achieved using an initial pulse having a pulse width of 1 μs or more followed by a plurality of pulses having a pulse width of less than 1 μs.

19. The method in accordance with claim 13, wherein the pulses of the waveform have an amplitude of 50V or less.

20. The method in accordance with claim 13, wherein the pulses have an effective pulse width of 500 μs or more.
